(19) United States
(12) Patent Application Publication
(10) Pub. No.: US 2023/0233531 A1
(43) Pub. Date: Jul. 27, 2023

(54) USE OF NON-STEROIDAL ANTI-INFLAMMATORY COMPOUNDS FOR TREATMENT OF INFLAMMATION

(71) Applicant: University of Houston System, Houston, TX (US)

(72) Inventor: Ke-He RUAN, Bellaire, TX (US)

(21) Appl. No.: 18/158,717

(22) Filed: Jan. 24, 2023

Related U.S. Application Data
(60) Provisional application No. 63/302,782, filed on Jan. 25, 2022.
(60) Provisional application No. 63/306,737, filed on Feb. 4, 2022.

(51) Int. Cl. A61K 31/423; A61P 29/00

(52) U.S. Cl. CPC A61K 31/423; A61P 29/00

(57) ABSTRACT

The present disclosure provides use of non-steroidal anti-inflammatory compositions as described herein for used in the treatment of inflammation, inflammatory-related diseases, pain and/or fever.

This invention claims benefit and priority to U.S. Provisional Application No. 63/302,782 filed on Jan. 25, 2022 and U.S. Provisional Application No. 63/306,737 filed on Feb. 4, 2022, which are both incorporated herein by reference in their entirety.

This invention was made with government support under HL056712 and HL079389 awarded by the National Institutes of Health. The government has certain rights in the invention.

### TECHNICAL FIELD

The present disclosure relates to the use of non-steroidal anti-inflammatory compositions as described herein for used in the treatment of inflammation, inflammatory-related diseases or disorders, pain and/or fever. The disclosed compositions contain, as an active ingredient, derivatives of 2-amino 4-nitrophenol and 2,4-diaminophenol having modifications of the functional groups at the position 1, —OH and position 2, —NH using benzoic acid derivatives. Such derivatives are shown to function to inhibit the activities of prostaglandin E₂ (PGE₂) and microsomal PGE₂ synthase-1 (mPGES-1).

### BACKGROUND

Inflammation is an adaptive response that is triggered by external and internal stimuli, and an uncontrolled inflammatory state is always associated with multiple pathological consequences including arthritis, autoimmune disorders, cardiovascular diseases and cancers. [1] Since the isolation of salicyclate and subsequently the discovery of aspirin (acetyl salicyclate), non-steroidal anti-inflammatory drugs (NSAIDs) have long been used worldwide owing to their efficacy in reducing pain, fever and other inflammation-related symptoms, by the action model of block cyclooxygenases (COXs) at the site of inflammation. [2,3] The COXs which are present in two forms, COX-1 and COX-2, are membrane-bound heme-containing glycoproteins, which are expressed abundantly in the ER membrane, [4] are the upstream enzymes of the arachidonic acid (AA) metabolism pathway. They can couple to various downstream synthases to convert the unstable substrate, prostaglandin H₂ (PGH₂) to a set of prostanoids, such as prostacyclin (PGI₂) synthase (PGIS), thromboxane A2 (TXA₂) synthase (TXAS), and non-inducible PGE₂ synthase. PGIS produces PGI₂ which is involved in vascular protection through anti-platelet aggregation and vasodilation. TXAS produces TXA₂, an endogenous anti-bleeding factor, and non-inducible PGE₂ synthases produce the basal level PGE₂ involved in gastrointestinal protection. [5-9] During the inflammation response, prostaglandin E2 (PGE₂), a pro-inflammatory prostanoid, is rapidly biosynthesized from AA under the catalysis of inducible COX-2 and microsomal PGE₂ synthase-1 (mPGES-1). A basal amount of PGE₂ is required for normal physiological functions, it is constitutively produced by constitutive COX-1 coupling with cytosolic PGE₂ synthase (cPGES), and microsomal PGES-2 (mPGES-2). The cPGES, mPGES-2, and mPGES-1 are three PGE₂ synthases, whereas, only the inducible mPGES-1 has been identified as directly related to pathogenic inflammation [10-12]. The other two PGE₂ synthases are not inducible and have less impact on the pathogenic process of inflammation [13].

NSAIDs, such as aspirin, Advil, Motrin and Celebrex are commonly used in the treatment of inflammation, pain and fever. Due to the inhibitory effect on the upstream COXs, which will also result in reduction of the production of other downstream enzyme-produced prostanoids, such as PGI₂, TXA₂ and the basal level PGE₂ many serious adverse effects are observed including gastrointestinal toxicities, bleeding disorders, cardiovascular risks, renal injuries, and hepatotoxicity as well as hypertension and others. [14,15]

A solution to effectively reduce inflammatory PGE₂ production without reducing prostacyclin (PGI₂) through COX-2 inhibition is currently unavailable. Accordingly, novel anti-inflammatory compositions, without the inherent side effects of COX-2 inhibitors, are greatly needed.

### SUMMARY

The present invention relates to methods of treating inflammation, inflammatory-related diseases, pain and/or fever using the anti-inflammatory compositions disclosed herein which contain, as active ingredients, one or more derivatives of 2-amino-4-nitrophenol and 2,4-diaminophenol (herein referred to as “derivative compounds”). Such derivative compounds include, for example, those having modification of the functional groups at the position 1, —OH and the position 2, —NH of 2-amino-4-nitrophenol and 2,4-diaminophenol using benzoic acid derivatives such as, for example, 4-(fluoro-, difluoro- or trifluoro-methyl)-benzoic acid; 4-(methyl-, dimethyl- or trimethyl)-benzoic acid; 4-(chloro-, dichloro, or trichloro-methyl)-benzoic acid, and 4-(phenyl, diphenyl- or tri-phenol)-benzoic acid to name a few.

As described in detail below, using an animal study with carrageenan-induced paw-inflammation as a model, compounds capable of inhibiting acute inflammation in vivo have been identified. Using two measurement approaches: volume of the edema, and paw thickness, induced by the inflammation, the compounds demonstrated similar effects as COX-2 inhibitor Celebrex on inhibition of the acute inflammation, but GI and heart insults were not observed. Thus, development of said compounds provides a new generation of NSAIDs that overcome the side-effects of the current nonselective NSAIDs and selective COX-2 inhibitors.

Accordingly, the present disclosure provides derivative compounds which can be used in the treatment of inflammation, inflammatory-related diseases, pain and/or fever wherein said treatments are administered without the side effects associated with current NSAIDS. While not wanting to be bound to any one particular theory, said derivatives are believed to act to inhibit the activities of PGE₂ and microsomal PGE₂ synthase-1 (mPGES-1) leading to a reduction in PGE₂ biosynthesis. In a non-limiting embodiment, the present disclosure provides for in vivo use of the compounds, 4-(R)-benzoic acid modified 2,4-diamonophenol and 2-amino, 4-nitrol phenol derivatives to prevent and treat inflammation, inflammatory-related diseases, pain and/or fever through inhibition of the downstream inducible mPGES-1. In an embodiment, the “R” group is substituted by a chemical group, such as 4-(fluoro-, difluoro- or trifluoro-methyl), 4-(methyl, dimethyl- or trimethyl), 4-(phenol-, diphenol- or triphenol) or 4-(chloro-, dichloro- or trichloro-methyl).

In further embodiments, pharmaceutical compositions comprising the derivative compounds and a pharmaceutical acceptable carrier are provided. Said pharmaceutical compositions can be used for preventing or treating inflammation, inflammatory-related diseases, pain and/or fever in a subject.

In an embodiment, disclosed derivative compounds and pharmaceutical compositions comprising said derivative compounds can be used for treating a subject suffering from inflammation, an inflammatory-related disease, pain and/or fever or a subject at risk for developing inflammation, an inflammatory-related disease, pain and/or fever, said use comprising administering to the subject, an effective amount of one or more of the derivative compounds disclosed herein in a pharmaceutically acceptable form. For such treatments, the administration of the derivative compound(s), through their inhibition of PGE₂ and mPGES-1, lead to a reduction in PGE₂ biosynthesis, thereby inhibiting or reducing the symptoms of inflammation.

In yet another embodiment, kits comprising the derivative compounds for treatment of inflammation, inflammatory-related diseases, pain and/or fever are provide. Such kits contain materials useful for the treatment of inflammation as described herein. The kits may comprise one or more of the following components: a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing inflammation, inflammatory-related diseases or disorders, pain and/or fever and may have a sterile access port (for example the container may be an intravenous solution bag or a vial having a stopper pierceable by a hypodermic injection needle).

### BRIEF DESCRIPTION OF THE FIGURES

In order to better understand the subject matter that is disclosed herein and to exemplify how it may be carried out in practice, embodiments will now be described, by way of non-limiting example, with reference to the accompanying drawings. With specific reference to the drawings, it is stressed that the particulars shown are by way of example and for purposes of illustrative discussion of embodiments of the disclosure.

FIG. 1A-B. FIG. 1A. Establishing an acute inflammation model on mouse paw. FIG. 1A. Carrageenan injection site on mouse paw; FIG. 1B. Monitoring of the effects of the compounds on reducing the carrageenan-induced paw inflammation.

FIG. 2A-B. Custom-made device for quantitative measurement of the carrageenan-induced paw inflammation/edema. FIG. 2A. Device made for the quantitative measurement of the paw inflammation/edema. FIG. 2B. Calculation formula.

FIG. 3A-B. Effects of the compounds on carrageenan-induced paw inflammation in BABL-c mice model. FIG. 3A. Effects of the compounds on carrageenan-induced paw inflammation measured by the volume quantitation. FIG. 3B. Effects of the compounds on carrageenan-induced paw inflammation measured by the paw thickness.

FIG. 4A-B. Effects of the compounds on carrageenan-induced paw inflammation in CD-1 mice model. FIG. 4A. Effects of the compounds on carrageenan-induced paw inflammation measured by the volume quantitation. FIG. 4B. Effects of the compounds on carrageenan-induced paw inflammation measured by the paw thickness.

FIG. 5A-B. Dose-dependent anti-inflammatory activity of 2-Me in CD-1 mice model. FIG. 5A. Dose-dependent anti-inflammatory activity of 2-Me. FIG. 5B. Different time points of dosage-dependent anti-inflammatory activities of 2-Me.

FIG. 6A-B. Dose-dependent anti-inflammatory activity of compound 3-F in CD-1 mice model. FIG. 6A. Dose-dependent anti-inflammatory activity of compound 3-F FIG. 6B. Different time points of dose-dependent anti-inflammatory activities of compound 3-F.

FIG. 7A-B. Effects of the lead compounds on reducing of the paw inflammation measured by paw thickness method. FIG. 7A. Effects of 2-Me on reducing of acute paw-inflammation measured by paw thickness method. FIG. 7B. Effects of 3-F derivative on reducing of acute paw-inflammation measured by paw thickness method.

### DETAILED DESCRIPTION

The present disclosure provides methods for treatment of inflammation, inflammatory-related diseases, pain and/or fever (“disclosed treatments”) to a subject in need, comprising administration of one or more of the anti-inflammatory compositions disclosed herein which contain, as active ingredients, derivatives of 2-amino-4-nitrophenol and 2,4-diaminophenol (herein referred to as “derivative compounds”). Such derivative compounds include, for example, those having modification of the functional groups at the position 1, —OH and the position 2, —NH of 2-amino-4-nitrophenol and 2,4-diaminophenol using benzoic acid derivatives such as, for example, 4-(fluoro-, difluoro- or trifluoro-methyl)-benzoic acid; 4-(methyl-, dimethyl- or trimethyl)-benzoic acid; 4-(chloro-, dichloro, or trichloro-methyl)-benzoic acid, and 4-(phenyl, diphenyl- or tri-phenol)-benzoic acid to name a few.

In one embodiment, a derivative compound for use in the disclosed treatments is one resulting from the modification of the position 1, —OH group (R1) and position 2 —NH group (R2) of 2-amino, 4-nitrol phenol resulting in products with anti-inflammatory properties and which are capable of inhibiting PGE₂ biosynthesis and activity, and mPGES-1 activity. The result is the compound of formula (I):

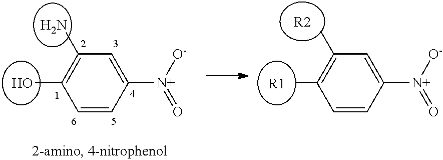

Compounds further include those where R1 and R2 are modified by benzoic acid resulting in products with anti-inflammatory properties and which are capable of inhibiting PGE₂ biosynthesis and activity, and mPGES-1 activity. The result is the compound of formula (III):

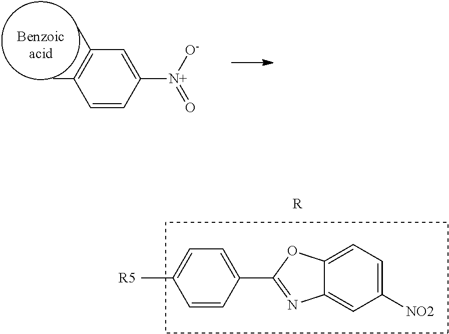

In an embodiment, derivative compounds for use in the disclosed treatments include those where the R5 is a fluoro-, difluoro- or trifluoro-group and wherein said derivative compounds exhibit anti-inflammatory activity and/or inhibit PGE₂ biosynthesis and activity, and mPGES-1 activity:

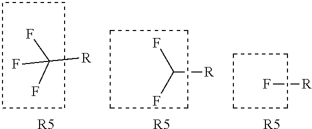

In another embodiment, derivative compounds for use in the disclosed treatments include those where the R5 is a methyl-, dimethyl- or trimethyl-group and wherein said derivative compounds exhibit anti-inflammatory activity and/or inhibit PGE₂ biosynthesis and activity, and mPGES-1 activity:

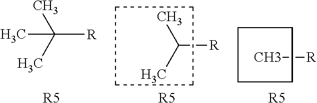

In another embodiment, derivative compounds for use in the disclosed treatments include those where the R5 is a chloro-, dichloro, or trichloro-group and wherein said derivative compounds exhibit anti-inflammatory activity and/or inhibit PGE₂ biosynthesis and activity, and mPGES-1 activity:

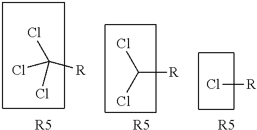

In another embodiment, derivative compounds for use in the disclosed treatments include those where the R5 of is a phenyl, diphenyl- or triphenyl-group and wherein said derivative compounds exhibit anti-inflammatory activity and/or inhibit PGE₂ biosynthesis and activity, and mPGES-1 activity:

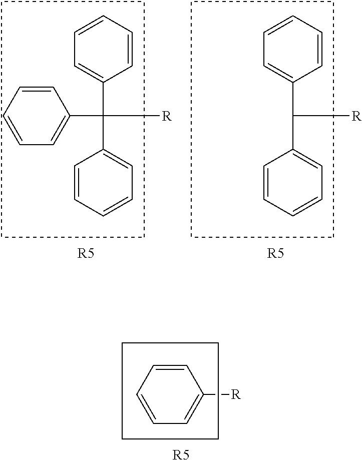

In another embodiment, derivative compounds for use in the disclosed treatments include those resulting from the modification of the position 1, —OH group (R1) and position 2 —NH group (R2) of 2,4-diaminophenol resulting in products with anti-inflammatory properties and which are capable of inhibiting PGE₂ biosynthesis and activity, and mPGES-1 activity. Compound of formula (II):

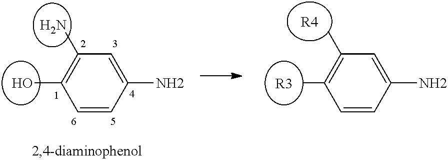

In another embodiment, derivative compounds for use in the disclosed treatments include those where R3 and R4 are modified by benzoic acid resulting in products with anti-inflammatory properties and which are capable of inhibiting PGE₂ activity and biosynthesis, and mPGES-1 activity. Compound of formula (IV):

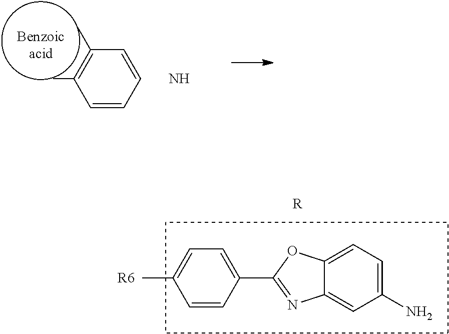

In an embodiment, derivative compounds for use in the disclosed treatments are provided where the R6 is a fluoro-, difluoro- or trifluoro-group and wherein said derivative compounds exhibit anti-inflammatory activity and/or inhibit PGE₂ biosynthesis and activity, and mPGES-1 activity:

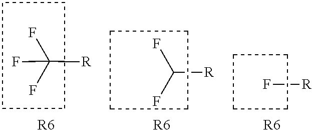

In another embodiment, provided derivative compounds for use in the disclosed treatments include those where the R6 is a methyl-, dimethyl- or trimethyl-group and wherein said derivative compounds exhibit anti-inflammatory activity and/or inhibit PGE₂ biosynthesis and activity, and mPGES-1 activity:

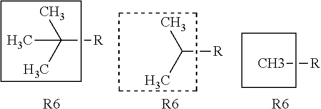

In another embodiment, provided derivative compounds for use in the disclosed treatments include those where the R6 of the compound is a chloro-, dichloro-, or trichloro-group and wherein said derivative compounds exhibit anti-inflammatory activity and/or inhibit PGE₂ biosynthesis and activity, and mPGES-1 activity:

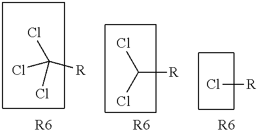

In another embodiment, provided derivative compounds for use in the disclosed treatments include those where the R6 is a phenyl, diphenyl- or triphenyl-group and wherein said derivative compounds exhibit anti-inflammatory activity and/or inhibit PGE₂ biosynthesis and activity, and mPGES-1 activity:

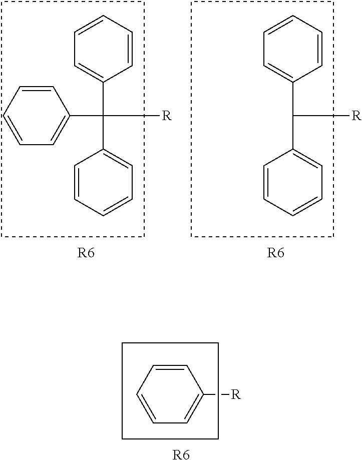

Methods for the synthesis of the derivative compounds provided herein having anti-inflammatory activity are provided. In an embodiment, a method is provided wherein derivatives of 2-amino 4-nitrophenol are derived using a method comprising a first step of mixing a benzoic acid derivative with 2-amino 4-nitrophenol under conditions wherein the benzoic acid reacts with the 2-amino 4-nitrophenol to form a derivative compound. In a second step of the method, the resulting reaction mixture may then be neutralized to a pH of about 7.0. In yet a further third step, the synthesis of derivatives of 2,4-diaminophenol is provided wherein the product derivatives of 2-amino 4-nitrophenol obtained in the first and second step are further reacted to convert —NO₂ of the derivatives of the 2-amino 4-nitrophenol to —NH₂ of the derivatives of 2,4-diaminophenol as final products.

Benzoic derivatives that may be used in the disclosed methods include, but are limited to, for example, 4-(fluoro-, difluoro- or trifluoro-methyl)-benzoic acid; 4-(methyl-, dimethyl- or trimethyl)-benzoic acid; 4-(chloro-, dichloro-, or trichloro-methyl)-benzoic acid, and 4-(phenyl, diphenyl- or tri-phenol)-benzoic acid to name a few.

In a specific, non-limiting embodiment, the synthesis of derivatives of 2-amino 4-nitrophenol is provided comprising the following steps: (Step 1): Polyphosphoric acid (PPA, Sigma-Aldrich) was first heated to 110° C. and 0.001-1 mol 2-amino-4 nitrophenol (Sigma-Aldrich) and 0.0015-1.5 mol (mol ratio of 1:1-1.5) corresponding benzoic acid (4-trifluoromethyl benzoic acid, Oakwood Chemical and 4-biphenylcarboxylic acid, Acros organics) were simultaneously added. The resulting mixture is then heated to 120-180° C. for 2-4 hours; (Step 2): At the end of the reaction, the solution is poured into ice-water and neutralized to pH 7.0. The precipitate is then filtered and collected as crude product.

In a non-limiting embodiment, a method for the synthesis of the derivatives of 2,4-diaminophenol comprises the additional (Step 3) wherein the product derivatives of 2-amino 4-nitrophenol as described above are further reacted using Step 3: The crude mid product was obtained by recrystallizing via boiling in ethanol. The crude mid product is heated in 20 ml ethanol with Tin (II) chloride (SnCl₂) at 70° C. for 10-16 hours. After the reaction, the mixture is cooled to room temperature and poured into ice-water. Saturated Sodium bicarbonate (NaHCO₃) is then used for neutralizing the mixture. Using an alternative step 3, the crude mid-product and 10% palladium on activated charcoal is first dissolved in methanol. Then the mixture was bubbled with hydrogen gas in room temperature for 2 hours to acquire the crude final product:

$$X - {NO}_{2} \frac{{H}_{2} , 10 \% palladium on activated charcoal}{Methanol , 2 hours} X - {NH}_{2}$$

This crude final product was then filtered to obtain the aqueous layer. The aqueous layer obtained from step 3, or alternative step 3, was then extracted twice with EtOAc (500 ml). The final combined organic layers were dried by anhydrous MgSO₄ and evaporated to obtain the final product (26,27).

With regard to the specific steps describe above, for production of the derivative compounds, it is understood that one skilled in the art may alter the conditions, e.g., temperatures and reaction times as well as the choice and concentrations of reagents while retaining the ability to successfully obtaining the derivative compounds of interest.

Provided herein is also a method of producing the derivative compounds in a form suitable for administration in vivo, the method comprising formulating the compounds with at least one pharmaceutically acceptable carrier, whereby a preparation of the derivative compound is formulated for administration in vivo.

Embodiments of pharmaceutical compositions comprise a therapeutically effective amount of one or more of the derivative compounds dissolved or dispersed in a pharmaceutically acceptable carrier. The preparation of a pharmaceutical composition that contains one or more of the derivative compounds and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Compositions include those that are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, i.e., a derivative compound, its use in the therapeutic or pharmaceutical compositions is contemplated.

Suitable examples of carriers and diluents are well known to those skilled in the art and include water-for-injection, saline, buffered saline, dextrose, water, glycerol, ethanol, propylene glycol, polysorbate 80 (Tween-80™), poly(ethylene)glycol 300 and 400 (PEG 300 and 400), pegylated castor oil (e.g. Cremophor EL), poloxamer 407 and 188, hydrophilic and hydrophobic carriers, and combinations thereof. Hydrophobic carriers include, for example, fat emulsions, lipids, pegylated phospholipids, polymer matrices, biocompatible polymers, lipospheres, vesicles, particles, and liposomes. Additional carriers include cornstarch, gelatin, lactose, sucrose, microcrystalline cellulose, kaolin, mannitol, dicalcium phosphate, sodium chloride, alginic acid, croscarmellose sodium, and sodium starch glycolate.

The composition may comprise different types of carriers depending on whether it is to be administered in solid, liquid or aerosol form, and whether it needs to be sterile for such routes of administration as injection. The derivative compounds described herein (and any additional therapeutic agent) can be administered by any method or any combination of methods as would be known to one of ordinary skill in the art (see, for example, Remington's Pharmaceutical Sciences, 18th Ed. Mack Printing Company, 1990, incorporated herein by reference).

Parenteral administration, in particular intravenous injection, may be used for administering of the compounds. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral compositions include those designed for administration by injection, e.g. subcutaneous, intradermal, intra-lesional, intravenous, intra-arterial, intramuscular, intrathecal or intraperitoneal injection. For injection, the compounds may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the compounds may be in powder form for constitution with a suitable vehicle, e.g., sterile pyrogen-free water, before use. Sterile injectable solutions are prepared by incorporating the compounds in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi.

For oral formulations, suitable pharmaceutically acceptable carriers include but are not limited to, alcohols, gum arabic, vegetable oils, benzyl alcohols, polyethylene glycols, gelate, carbohydrates such as lactose, amylose or starch, magnesium stearate, talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical compositions can be sterilized and if desired mixed with auxiliary agents, e.g., lubricants, disintegrants, preservatives, stabilizers, wetting agents, emulsifiers, salts for influencing osmotic pressure buffers, coloring, flavoring and/or aromatic substances and the like.

The compositions intended for oral use may be prepared according to any method known in the art and such compositions may contain one or more agents selected from the group consisting of inert, non-toxic pharmaceutically acceptable excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated, or they may be coated by known techniques for elegance or to delay release of the active ingredients. Formulations for oral use may also be presented as hard gelatin capsules wherein the active ingredient is mixed with an inert diluent. The oral dosage forms may be in the form of tablets (sustained release and/or immediate release), troches, lozenges, powders, or granules, hard or soft capsules, microparticles (e.g., microcapsules, microspheres and the like), buccal tablets, suppositories, solutions, suspensions, etc.

The formulations containing one or more of the derivative compounds may be designed to be short-acting, fast-releasing, long-acting, or sustained-releasing as described herein. Thus, the pharmaceutical formulations may also be formulated for controlled release or for slow release.

Sustained release formulations are provided which preferably slowly release the compound derivatives, when ingested and exposed to gastric fluids, and then to intestinal fluids. The dosage forms may optionally be coated with one or more materials suitable for the regulation of release or for the protection of the formulation. In one embodiment, coatings are provided to permit either pH-dependent or pH-independent release. A pH-dependent coating serves to release the active in desired areas of the gastro-intestinal (GI) tract, e.g., the stomach or small intestine, such that an absorption profile is provided which can provide treatment over a period of, for example, at least about eight hours and preferably about twelve hours to up to about twenty-four hours of analgesia to a patient. When a pH-independent coating is desired, the coating is designed to achieve optimal release regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Suitable examples of sustained-release preparations include semipermeable matrices of solid hydrophobic polymers containing the compound derivatives, which matrices are in the form of shaped articles, e.g., films, or microcapsule. Examples of sustained-release matrices include polyesters, hydrogels (for example, poly (2-hydroxyethyl-methacrylate), or poly (vinyl alcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot™ (injectable microspheres composed of lactic acid-glycolic acid copolymer and leuprolide acetate), and poly-D-(−) hydroxybutyric acid. While polymers such as ethylene-vinyl acetate and lactic acid-glycolic acid enable release of molecules for over 100 days, certain hydrogels release proteins for shorter time periods.

In another embodiment, pharmaceutical compositions are provided comprising an aerosol of a dispersion of particles, wherein the particles comprise a derivative compound and an additive that enhances absorption of the drug into tissue of the respiratory system and administering the aerosol to the respiratory system of the subject. Pulmonary drug delivery of the derivative compound is accomplished by inhalation of an aerosol through the mouth and throat. In an embodiment, the pharmaceutical compositions may be formulated for pulmonary delivery. Optimized formulations for such delivery may include addition of permeability enhancers (mucoadhesives, nanoparticles, and the like) as well as combined use with a pulmonary drug delivery device (for example, one that provides controlled particle dispersion with particles aerosolized to target the upper nasal cavity)

Pharmacologically active amounts of the drug substance, i.e., derivative compound, are thereby delivered to the circulation or directly to a site of action. The present disclosure relates to a dosage form (for example a nasal spray, a nasal gel, a nasal ointment, inhalation solutions, inhalation suspensions, inhalation sprays, dry powder or an aerosol) which is specifically designed or adapted for administration of a drug substance to the nasal structures.

Also provided is a nanoparticle comprising one or more derivative compounds for use in treatment of inflammation, or diseases or disorders associated with inflammation. Such nanoparticles can be natural or synthetic and may be incorporated into a pharmaceutical composition. They can be created from biological molecules or from non-biological molecules. In particular embodiments, the nanoparticle is formed from a biocompatible polymer. Examples of biocompatible polymers include polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, or combinations thereof. In some cases, the nanoparticle is formed from a polyethylene glycol (PEG), poly(lactide-co-glycolide) (PLGA), polyglycolic acid, poly-beta-hydroxybutyrate, polyacrylic acid ester, or a combination thereof. In a specific embodiment the nanoparticle is a nanoliposome. Such nanoliposomes may be composed of phospholipids such as 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1,2-distearoyl-sn-glycero-3-phospho-(1′-rac-glycerol) (DSPG), 1,2-dipalmitoyl-sn-glycero phospho-(1′-rac-glycerol) (DPPG), 1,2-dimyristoyl-sn-glycero-3-phospho-(1′-rac-glycerol) (DMPG), 1,2-dioleoyl-sn-glycero-3-phospho-(1′-rac-glycerol) (DOPG), dipalmitoyl phosphatidylserine (DPPS), distearoyl phosphatidylserine (DSPS), dipalmitoyl phosphatidylinositol (DPPI), distearoyl phosphatidylinositol (DSPI), dipalmitoyl phosphatidic acid (DPPA), distearoyl phosphatidic acid (OSPA), 1,2-diacyl-3-trimethylammonium-propanes, (including but not limited to, dioleoyl (DOTAP), 1,2-dipalmitoyl-sn-glycero-3-phosphoethanolamine-N [methoxy(polyethylene glycol)-2000] (DPPE-PEG2000), 1,2-distearoyl-sn-glycero-3-phosphoethanolamine-N-[methoxy(polyethylene glycol)-1000] (DSPE-PEG2000), and cholesterol.

In some embodiments, the one or more derivative compound is coated on the nanoparticle using a crosslinking agent. In some embodiments, the derivative compound is adsorbed onto the nanoparticle surface. In some embodiments, the derivative compound is adsorbed onto the nanoparticle surface followed by covalent crosslinking of the derivative compound to the nanoparticle surface using a crosslinking agent.

Crosslinking agents suitable for crosslinking the derivative compound to produce the nanoparticle, or coat the derivative compound on the nanoparticle are known in the art, and include those selected from the group consisting of formaldehyde, formaldehyde derivatives, formalin, glutaraldehyde, glutaraldehyde derivatives, a protein cross-linker, a nucleic acid cross-linker, a protein and nucleic acid cross-linker, primary amine reactive crosslinkers, sulfhydryl reactive crosslinkers, sulfydryl addition or disulfide reduction, carbohydrate reactive crosslinkers, carboxyl reactive crosslinkers, photoreactive crosslinkers, cleavable crosslinkers, AEDP, APG, BASED, BM(PEO)3, BM(PEO)4, BMB, BMDB, BMH BMOE, BS3, BSOCOES, DFDNB, DMA, DMP, DMS, DPDPB, DSG, DSP, DSS, DST, DTBP, DTME, DTSSP, EGS, HBVS, sulfo-BSOCOES, Sulfo-DST, and Sulfo-EGS.

Pharmaceutical compositions comprising i the incorporated derivative compounds may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients, or auxiliaries which facilitate processing of the proteins into preparations that can be used pharmaceutically. Proper formulation is dependent upon the route of administration chosen.

The derivative compounds may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

In one embodiment, the present disclosure provides a method of treating, preventing the progression of, or delaying the onset of inflammation, an inflammatory-related disease, pain and/or fever in a subject, the method comprising administering to the subject a therapeutically effective amount of at least one derivative compound. In one embodiment, such treatments are designed to reduce the expression and/or activity of PGE₂ and/or mPGES-1 in the subject to be treated.

A “subject” can include a human subject for medical purposes, such as for the treatment of inflammation, an inflammatory-related disease, pain and/or fever or the prophylactic treatment for preventing the onset of inflammation, an inflammatory-related disease, pain and/or fever, or an animal subject for medical, veterinary purposes, or developmental purposes. Further, a “subject” can include a patient afflicted with or suspected of being afflicted with inflammation, an inflammatory-related disease, pain and/or fever.

Thus, the terms “subject” and “patient” are used interchangeably herein. In one embodiment, a method is provided of treating or preventing inflammation, an inflammatory-related disease, pain and/or fever in a subject in need thereof, the method including administration to the subject of a therapeutically effective amount of a derivative compound. In an embodiment, a subject is a mammal. In another embodiment the subject is a human.

Since the disclosed compositions contain the derivative compounds, or a salt thereof as an active ingredient, they can inhibit inflammatory responses even at an early stage and exhibit a potent anti-inflammatory effect by regulating the expression and/or activity of PGE₂ and/or mPGES-1. Specifically, the derivative compounds and compositions of the present disclosure can inhibit an early stage of inflammatory response by inhibiting the expression and/or activity of PGE₂ and/or mPGES-1. Based on the anti-inflammatory effect of the derivative compounds described herein, the compositions of the present invention may be compositions for preventing, ameliorating or treating inflammation, an inflammatory-related disease, pain and/or fever.

Such “inflammatory diseases” are not limited in the kind thereof but may be selected from the group consisting of inflammatory lung disease, inflammatory liver disease, inflammatory bowel disease, autoinflammatory disease, inflammatory central nervous system disease, inflammatory skin disease, and allergic inflammatory disease. More specifically, the inflammatory disease may be selected from the group consisting of interstitial lung disease (ILD), non-alcoholic steatohepatitis (NASH), Crohn's disease, ulcerative colitis, rheumatoid arthritis, type 1 diabetes, lupus, multiple sclerosis, Parkinson's disease, scleroderma and psoriasis.

Such, “inflammation” means that the subject has symptoms typically associated with inflammation. Such symptoms may be attributable to the presence in the subject of an inflammatory disease or injury to the subject. Such inflammation symptoms include, for example, fever, chills, fatigue/loss of energy, headaches, loss of appetite, muscle stiffness, and redness and swelling at a site of injury.

As used herein, the terms “treat,” treating,” “treatment,” and the like, are meant to decrease, suppress, attenuate, diminish, arrest, the underlying cause of the inflammation or to stabilize the development or progression of an inflammatory disease and/or symptoms associated therewith. The terms “treat,” “treating,” “treatment,” and the like, as used herein can refer to curative therapy, prophylactic therapy, and preventative therapy. Accordingly, as used herein, “treating” means either slowing, stopping or reversing the progression of inflammation or inflammatory disease, including reversing the progression to the point of eliminating the symptoms of the inflammation or inflammatory disease.

As used herein, the terms “prevent,” “preventing,” “prevention,” “prophylactic treatment” and the like refer to reducing or inhibiting the probability of developing symptoms of inflammation in a subject, who does not have, but is at risk of or susceptible to developing inflammation or an inflammatory disease. Thus, in some embodiments, a derivative compound can be administered prophylactically to prevent the onset of inflammation or to prevent the recurrence of inflammation in a subject.

As used herein, the term “inhibit” or “inhibits” means to decrease, suppress, attenuate, diminish, arrest, or stabilize the development or progression of inflammation by at least 10%, 20%, 40%, 40%, 50%, 60%, 70%, 80%, 90%, 95%, 98%, 99%, or even 100% compared to an untreated control subject. By the term “decrease” is meant to inhibit, suppress, attenuate, diminish, arrest, or stabilize a symptom of inflammation. It should be appreciated that treating a disease, disorder or condition does not require that the disease, disorder, condition, or symptoms associated therewith be completely eliminated.

Toxicity or efficacy of the compositions to elicit their anti-inflammatory effect can be determined by standard procedures in cell cultures or experimental animals. Data obtained from cell culture assays and laboratory animal studies can be used in formulating a range of dosage for use in humans. The dosage of such components lies, for example, within a range of administered concentrations that include efficacy with little or no toxicity. The dosage may vary within this range depending upon the dosage form employed and the route of administration utilized.

Any of the derivative compounds, provided herein may be used in therapeutic methods described herein. For use in the therapeutic methods described herein, the compounds would be formulated, dosed, and administered in a fashion consistent with good medical practice. Factors for consideration in this context include the particular disorder being treated, the particular subject being treated, the clinical condition of the subject, the cause of the disease or condition, the site of delivery of the agent, the method of administration, the scheduling of administration, and other factors known to medical practitioners or those of skill in the art.

For the treatment of inflammation, an inflammatory-related disease, pain and/or fever the appropriate dosage of the derivative compounds (when used alone or in combination with one or more other additional therapeutic agents) will depend on the type of disease to be treated, the route of administration, the body weight of the patient, the severity and course of the disease, whether the compound is administered for preventive or therapeutic purposes, previous or concurrent therapeutic interventions, the patient's clinical history and response to the compound, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The disclosed compositions may be used in methods of treatment alone or in combination with surgery, radiation therapy, hormone therapy, chemotherapy and a biological response modulator. In certain embodiments, the presently disclosed subject matter also includes combination therapies. Additional therapeutic agents, which are normally administered to treat or prevent inflammation, an inflammatory-related disease, pain and/or fever may be administered in combination with a derivative compound as disclosed herein. For example, the derivative compound may optionally be administered in conjunction with other compounds (e.g., therapeutic agents) or treatments useful in treating inflammation, an inflammatory-related disease, pain and/or fever. These additional agents may be administered separately, as part of a multiple dosage regimen, from the composition comprising a derivative compound as disclosed herein. Alternatively, these agents may be part of a single dosage form, mixed together with a derivative compound, in a single composition.

By “in combination with” is meant the administration of a derivative compound, with one or more therapeutic agents either simultaneously, sequentially, or a combination thereof. Therefore, a subject can be administered a combination of a derivative compound and one or more therapeutic agents at the same time (i.e., simultaneously) or at different times (i.e., sequentially, in either order, on the same day or on different days), so long as the effect of the combination of both agents is achieved in the subject. Where the derivative compound and one or more therapeutic agents are administered simultaneously, they can be administered to the subject as separate pharmaceutical compositions, each containing either a derivative compound or one or more therapeutic agents or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times. In such combination therapies, the therapeutic effect of the first administered agent is not diminished by the sequential, simultaneous, or separate administration of the subsequent agent(s).

The presently disclosed pharmaceutical compositions can be administered using a variety of methods known in the art. More particularly, as described herein, the compound derivatives can be administered to a subject for treatment of inflammation, an inflammatory-related disease, pain and/or fever by any suitable route of administration, including orally, nasally, transmucosally, parenterally, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections, intracisternally, topically, as by powders, ointments, including buccally and sublingually, transdermally, through an inhalation spray, or other modes of delivery known in the art.

A therapeutically effective dose can be estimated initially from in vitro assays, such as cell culture assays. A dose can then be formulated in animal models to achieve a circulating concentration range that includes the IC₅₀ as determined in cell culture. Such information can be used to determine useful doses more accurately in humans. Initial dosages can also be estimated from in vivo data, e.g., animal models, using techniques that are well known in the art.

One typical dosage would be in the range from about 0.0001 to 1000 mg/kg. The daily dose of the compound or salt thereof contained in the composition of the present invention may be about 0.0001 to 500 mg/kg of body weight, or 0.001 to 50 mg/kg of body weight. In other non-limiting examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein.

One having ordinary skill in the art could readily optimize administration to humans based on animal data. and the dose thereof may vary depending on the subject's body weight, age, sex, health condition, diet, the period of administration, the mode of administration, excretion rate, and the severity of the disease.

An initial higher loading dose, followed by one or more lower doses may be administered. However, other dosage regimens may be useful. The progress of this therapy is easily monitored by conventional techniques and assays. The compounds of certain embodiments will generally be used in an amount effective to achieve the intended purpose. For use to treat or prevent a disease condition, the derivative compounds, or pharmaceutical compositions thereof, are administered or applied in a therapeutically effective amount. Determination of a therapeutically effective amount is well within the capabilities of those skilled in the art, especially in light of the detailed disclosure provided herein.

The treatment, administration, or therapy can be continuous or intermittent. Continuous treatment, administration, or therapy refers to treatment on at least a daily basis without interruption in treatment by one or more days. Intermittent treatment or administration, or treatment or administration in an intermittent fashion, refers to treatment that is not continuous, but rather cyclic in nature. Treatment according to the presently disclosed methods can result in complete relief or cure from inflammation or partial amelioration of one or more symptoms of inflammation and can be temporary or permanent. The compositions disclosed herein may be administered once or several times a day.

The compound containing compositions may be administered by an initial bolus followed by a continuous infusion to maintain therapeutic circulating levels of drug product. As another example, the inventive compound may be administered as a one-time dose. Those of ordinary skill in the art will readily optimize effective dosages and administration regimens as determined by good medical practice and the clinical condition of the individual patient.

The attending physician for patients treated with the compounds of certain embodiments would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

The compounds described herein may be administered in combination with one or more other agents or “therapeutic agents” for use in treatment of inflammatory diseases and symptoms associated with said inflammation, e.g., pain and fever. A composition may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other.

There are a variety of drugs prescribed for patients with inflammatory diseases and symptoms of inflammation. It's important for both patients living with such diseases and those who care for them to understand the prescribed medication, to follow the directions of usage, and to be able to recognize the possible side effects associated with the medicine.

In another aspect of the embodiment, an article of manufacture (e.g., a kit) containing materials useful for the treatment of inflammatory diseases and symptoms associated with inflammation as described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

The label or package insert indicates that the composition is used for treating the condition of choice. The article of manufacture may comprise (a) a first container with a composition contained therein, wherein the composition comprises the compounds; and (b) a second container with a composition contained therein, wherein the composition comprises a further therapeutic agent.

Kits in certain embodiments may further comprise a package insert indicating that the compositions can be used to treat a particular condition. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example

### Materials and Methods

Drugs and Chemicals. Celebrex and lambda-carrageenan were purchased form Sigma Aldrich (USA) and used without purification. The tested compound 2-Me, is represented by Formula (II)

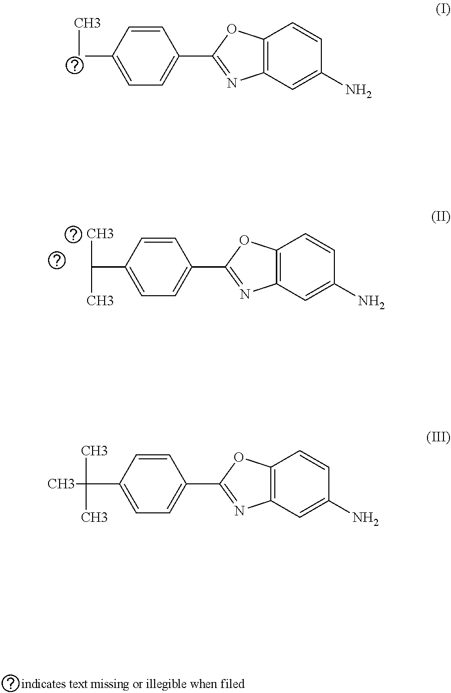 [SMILES: CCc1ccc(-c2nc3cc(N)ccc3o2)cc1 ; CC(C)c1ccc(-c2nc3cc(N)ccc3o2)cc1 ; CC(C)(C)c1ccc(-c2nc3cc(N)ccc3o2)cc1]

The tested compound, 3-F, is represented by Formula (IX).

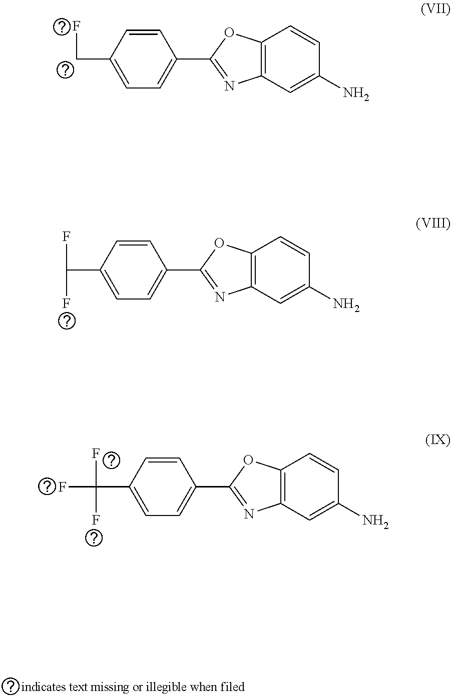 [SMILES: Nc1ccc2oc(-c3ccc(CF)cc3)nc2c1 ; Nc1ccc2oc(-c3ccc(C(F)F)cc3)nc2c1 ; Nc1ccc2oc(-c3ccc(C(F)(F)F)cc3)nc2c1]

Animals. The adult CD-1 mice (18-20 g) and BABL-c (18-20 g) were purchase from Envigo Plus vendor (USA) and used in this study. The mice were hosted under standard condition (23±2° C.; 12 h/12 h light/dark cycle; 50% humidity). The protocol was approved by Institutional Animal Care and Use Committee (IACUC). And all procedures were conducted according to IACUC. After all the experiments, the animals were euthanized with CO₂ under the procedure of IACUC.

Injections of compounds. The synthesized compounds to be tested were injected 10 mins before carrageenan injection. All the compounds were freshly prepared. All the compounds were dissolved in PBS and totally dissolved after ultrasonic. The compounds were injected with 26-G sterile needle via intramuscular injection. After immobilizing the mice, the hind limb was immobilized with the little finger and the then, the injection site was disinfected with 70% alcohol, and the needle was inserted into the root of the hindlimb at an angle of 45 degrees. The needle plug was withdrawn to make sure there was no injection into the blood vessel. If there was no blood return, the compounds were injected slowly. The mice were place back to their cages for 10 mins before the next step.

Procedures for establishing of paw inflammation. The procedure of establishing inflammation mice model and the procedure of measurement of paw edema were describe as follow: 1% lambda-carrageenan was freshly prepared before injection. Carrageenan powder was first dissolved in cold PBS and vortexed for 5 times. Then the mixture was placed in 50° C. water for 5 mins until all the milky white matrix dissolved completely. A 26 G*⅜ (0.45 mm*10 mm) sterile needle was used for injection. Carrageenan was subcutaneous injected slowly into the middle of the mice left hind paw. The time of injection Carrageenan was count as the start time point (0 h) and the paw swelling size was recorded as paw thickness and paw edema volume.

### Results

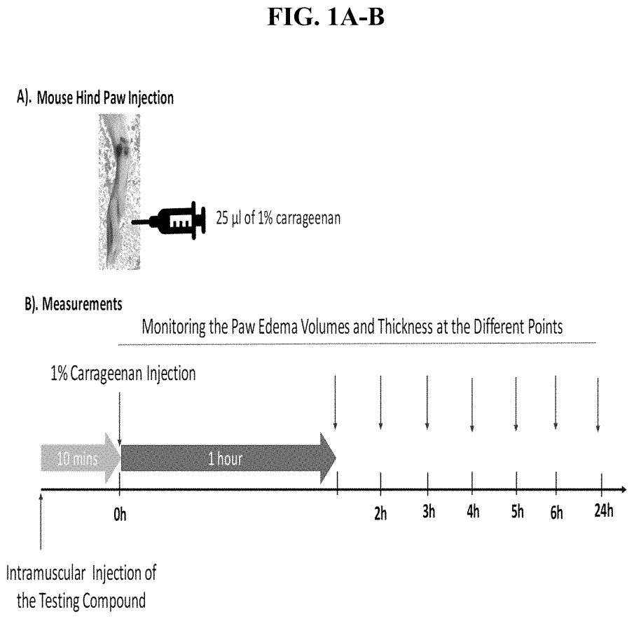

Establishing of Carrageenan-induced Inflammatory Paw Edema Mice Model. To examine the anti-inflammatory characteristic of the tested compound 2-Me (Formula II) and its representative derivative, 3-F (Formula IX) in vivo, the carrageenan-induced paw inflammation/edema in mice was used as an animal model. The carrageenan-induced paw edema inflammation is a well-defined model of acute inflammation. [19] It is widely and commonly used for evaluating the effectiveness of inhibitors of prostaglandin synthesis. Carrageenan is a complex of polysaccharides that functions in stimulating the expressions of COX-2 and mPGES-1 to release the proinflammatory mediator, PGE2, and its downstream factors, such as bradykinin, histamine, tachykinins, reactive oxygen, and nitrogen species. [20] In this study, to induce acute inflammation, a single dose of 25 μl of 1% solution of λ-carrageenan was injected subcutaneously into the pad area of the hairless skin on the underside of the left hind paw (FIG. 1A). The injection was performed using a 26-gauge needle targeting the center of the plantar region. The administration of leading compounds and the positive control group, Celebrex, and the negative control saline occurred 10 minutes prior to the λ-carrageenan intramuscular (IM) injection. To obtain the anti-inflammatory data, the animal condition was monitored hourly and paw-edema volume and thickness were measured hourly from 0 h to 6 h and again at 24 h, the injection of λ-carrageenan was counted as 0 h (FIG. 1B). After 24 h, local blood was collected from the hind paw of the mice for the next step assay to determine the PGE₂ and PGI₂ levels. Tissue samples, including the stomach and kidney, were collected after euthanization. The BABL-c mice were arranged into three groups: A. Control (Ctl) group: 50 μl saline; B. 2Me group: 50 mg/kg, 50 ul; C. 3-F group: 50 mg/kg, 50 ul. the CD-1 mice were arranged into 6 groups: A. Control (Ctl) group: saline 50 ul; B. 2-Me group: 50 mg/kg, 50 ul; C. 3-F group: 50 mg/kg, 50 ul; D: 2-Me group: 10 mg/kg, 50 ul; E. 3-F group: 10 mg/kg, 50 ul; F. Celebrex group: 50 mg/kg, 50 ul.

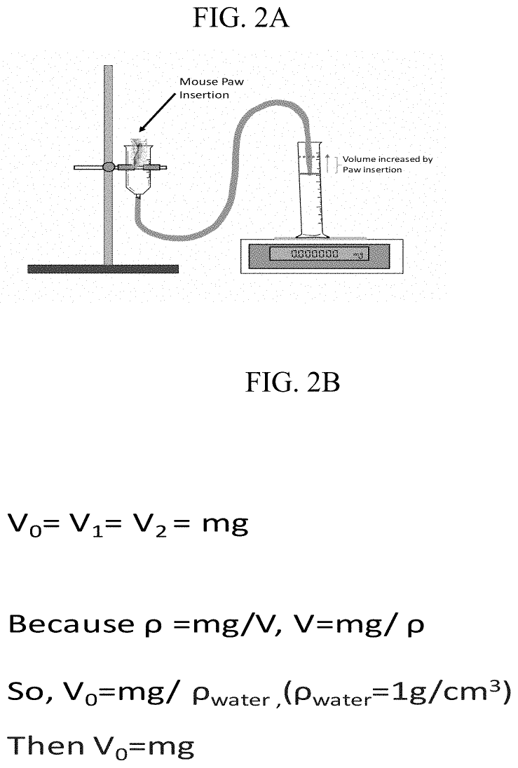

Establishing measurements of the carrageenan-induced acute inflammation/edema. To evaluate the anti-inflammatory effect of the tested compounds, two methods were used in the studies. In the model of acute inflammation induced by injection of carrageenan, the inflammation and PGE₂ level was indicated by the swelling of mice paw. As mentioned in FIG. 1B, edema could be assessed by measurement of paw thickness in the dorsal plantar axis by vernier caliper. The measurement point was pre-marked with an indelible pen and the thickness of the paw was recorded every hour. Paw thickness increase was calculated as the mean difference of paw thickness (A paw thickness/starting thickness of the corresponding paw). [21] Another method in this study is plethysmometry which is more rapid and accurate. [22] The paw edema of the inflammatory site is a three-dimensional volume change; the degree of edema should not be considered solely on a single cross-section. In order to measure the accurate volume of the left hind paw, a device based on plethysmometry was established (FIG. 2A). The two chambers are connected by a thin tube to form a connector which allows the liquid surface on both sides is always on the same horizontal line. The chamber on the left is firmly fixed on the iron frame, and the chamber on the right is placed on a high-precision electronic balance. The mice paw was inserted into the left chamber until the liquid surface arrived at the pre-marked indelible line. Then the liquid volume increase in the right chamber until the surface arrived at the same high as the left. The weight shown in the balance indicates the volume of the paw as calculated in FIG. 2B.

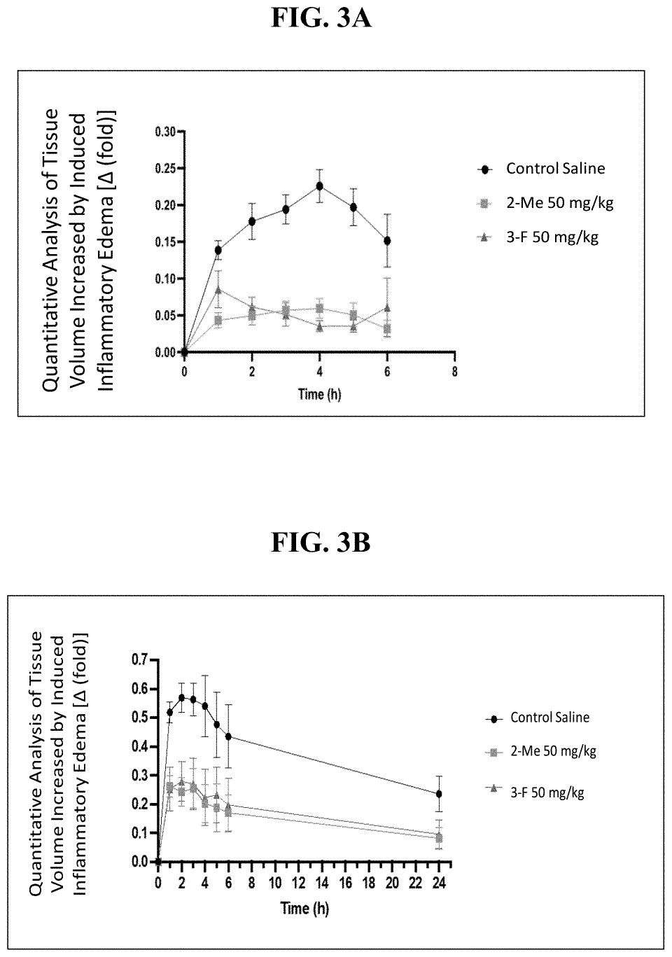

Anti-Inflammatory Activity of the tested compounds on carrageenan-induced paw inflammation on BABL-c Mice model. By the mean of the two methods described above, experiments were performed to examine the anti-inflammatory effect of the lead compounds. The in vivo activity was first tested in BABL-c mice model. As shown in FIG. 3A, administration of 50 mg/kg of 2-Me and 3-F significantly decreased the inflammatory edema similar to that of positive control of COX-2 inhibitor compared to the control group. The curve panel of 3-F shows the volume increased during the first 2 hours but decreased within the next 4 hours. The curve of 2-Me shows that the volume increased slightly in the first 4 hours and decreases in the following 2 hours. The paw thickness result shown the similar panels that comparing to the control group, both 2-Me and 3-F have significant anti-inflammatory potency. These results indicate that the tested compounds have potent anti-inflammation effects on the mouse model.

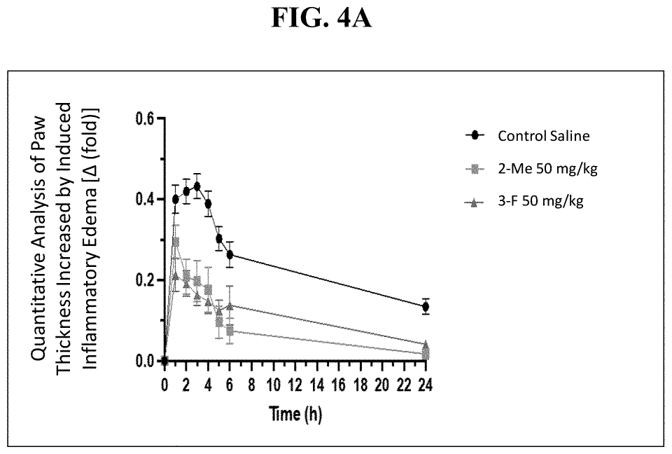

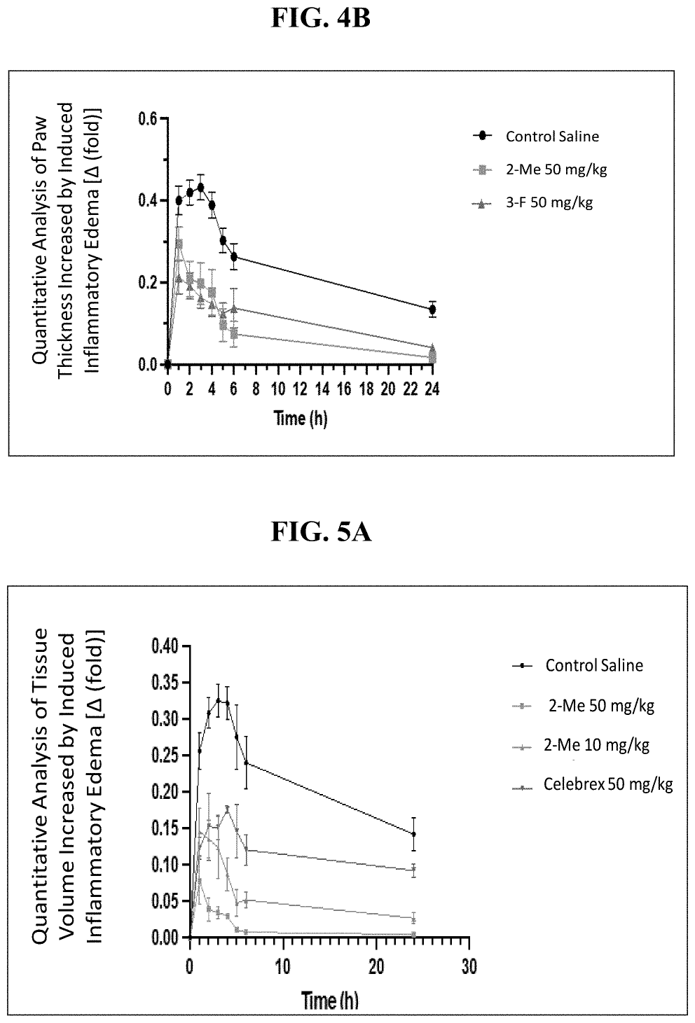

Anti-Inflammatory Activity of the tested compounds on carrageenan-induced paw inflammation on CD-1 Mice model. To further confirm the test compounds' potent anti-inflammation effects, another mice model was used, CD-1 mice, to perform these experiments. As shown in FIG. 4A-B, administration of 50 mg/kg 2-Me and 3-F significantly decrease the paw volume increase and thickness increase in CD-1 inflammation mice model. Compared to the control group, 50 mg/kg of the 2-Me and 3-F could reverse the inflammation-induced paw swelling. These findings further support that the tested compounds have activity to specifically inhibit mPGES-1 and have significant anti-inflammatory effects on the mouse model.

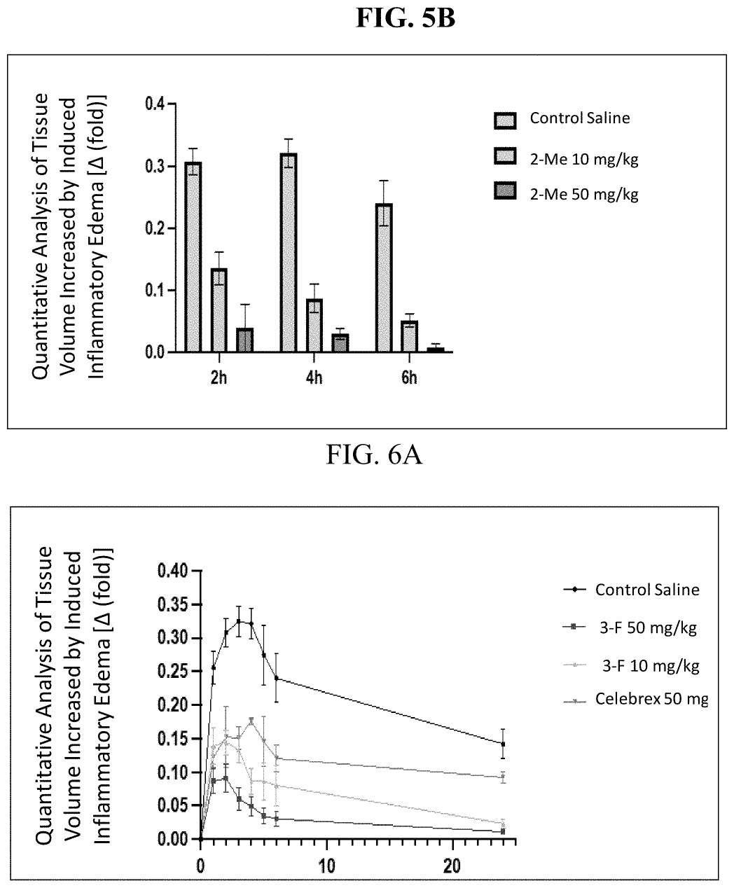

Dose-dependent Anti-Inflammatory Activity of 2-Me on CD-1 Paw Edema. More information of anti-inflammatory activity of the test compounds would help to establish a great degree of accuracy on their characteristics. By administration of the different dose of 2-Me, a curve was successfully obtained of dosage-dependent anti-inflammatory activity of 2-Me in the CD-1 mice model. 50 mg/kg of Celebrex was used as a positive control. The results showed that, compared to both the negative and positive control groups, increasing the dosage of 2-Me from 10 mg/kg to 50 mg/kg also improved its anti-inflammation activity (FIG. 5A). This finding could also be more intuitively understood by comparing the volume increase of the edema at different time points as shown in FIG. 5B. At 2, 4, and 6 h, increasing the concentration of compound could decrease the quantity of volume increase. These findings help to enhance the understanding the anti-inflammatory characteristic of 2-Me which acts in a dosage-dependent manner.

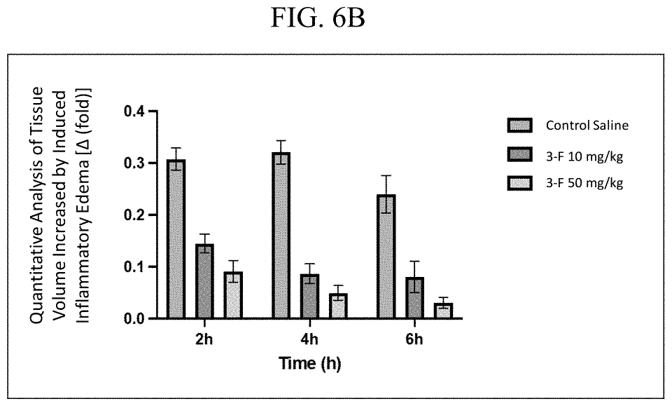

Dose-dependent Anti-Inflammatory Activity of 3-F on CD-1 Mouse-Paw Edema. The same experiment was also performed by using 3-F in CD-1 mice model. As the result shown in FIG. 6A and FIG. 6B, 3-F have a similar curve panel with 2-Me that shows an increasing potent anti-inflammatory effect corresponding with the dosage increase. This may be because they have a similar core structure and belong to the same chemical derivative.

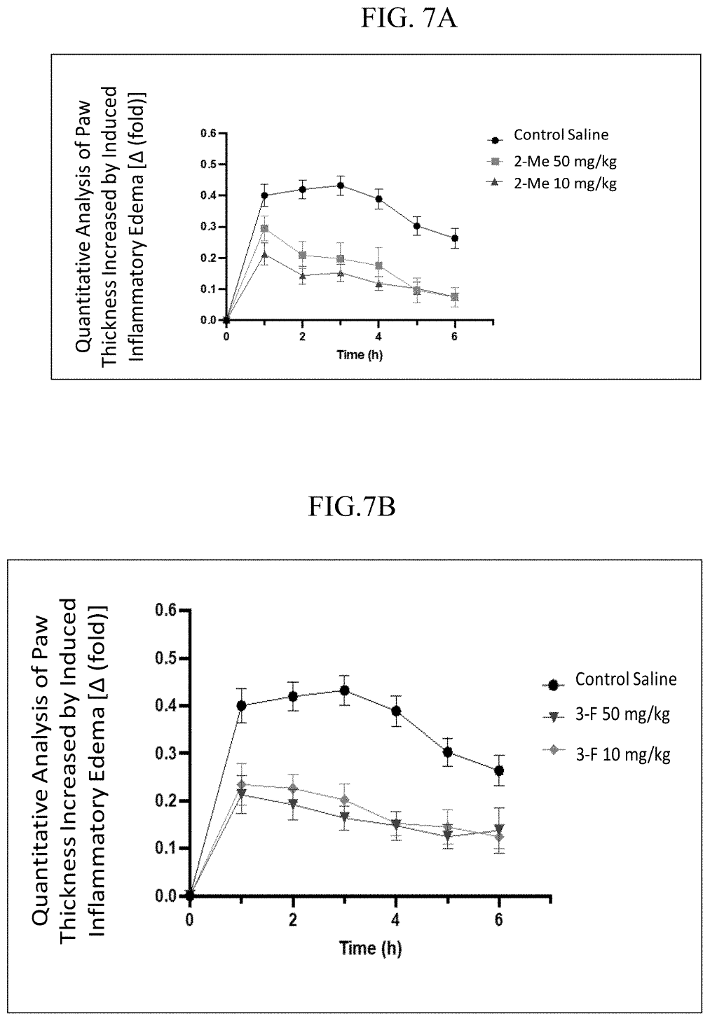

Dose-dependent Anti-Inflammatory Activity of 2-Me and 3-F on CD-1 on Paw thickness. At the same time, the paw thickness after administration of the leading compounds under different dosages were also recorded (FIG. 7A and FIG. 7B). Consistent with the dosage-dependence results of paw edema, increasing the concentration of these compounds also improve the anti-inflammatory effects as measured by paw thickness.

There is an urgent need for new anti-inflammatory drugs with less the adverse effects in clinical practice. Developing drugs which inhibit mPGES-1 without affecting other COX downstream synthases to only decrease PGE₂ level at inflammation site is an ideal therapeutic strategy to treat inflammatory diseases. [15,16] By successfully establishing a system of single-chain Enzymelinks of the upstream COX-2 and the downstream mPGES-1 thread by a 10 amino acid (AA) bio-linker (COX-2-10aa-mPGES-1 [9,11]), one can mimic endogenous inflammatory PGE₂ biosynthesis without affecting COX-2 coupling to other enzyme synthases, for example, PGIS. [17] This kind of Enzymelink has a kinetic advantage for converting the initial substrate, AA, to pro-inflammatory PGE₂ via a triple catalytic reaction due to the shorter distance between the catalytic domain of the two enzymes within a single polypeptide chain. By this method, it was possible to reduce the interference with the mPGES-1 inhibitor screen, such as the nonspecific inhibition and cross-inhibition of PGIS, by avoiding the use of the unstable intermediate PGH₂ as the initial substrate for PGE₂ biosynthesis. At the same time, another Enzymelink, COX-2-10aa-PGIS was established [14,18] in order to verify that the screen compound will not affect PGI₂ synthesis. By high through-put screening the compounds from a 380,000-compound library via this two Enzymelink systems, specific lead compounds were found for exclusive inhibition of COX-2 coupled to mPGES-1 to produce PGE₂ that does not have the cross-inhibition of PGIS producing PGI₂. By further modification and optimization of this structure, a set of compounds which target inflammation-induced mPGES-1 were established. As described above the inhibitory characteristic of these compounds, 3-F and 2-Me, on mPGES-1 was studied in different mice models. These compounds were found to be highly selective for mPGES-1 and to have potent anti-inflammatory effects in vivo. The results from this study indicate that the tested compounds may be successfully developed into novel anti-inflammatory drug with less site effects compared to that of the current NSAIDs. Increase of PGE₂ expression is highly associated with inflammation. Inducible mPGES-1 is the key enzyme which is coupled with COX-2 to produce PGE₂ at the inflammation site with the external and internal pathogen-stimuli. The Carrageenan-induced inflammation model is considered as an acute inflammation model in which expression of multiple inflammatory factors increase, such as PGE₂. According to the result of this study, the tested compounds reversed the paw thickness increase as compared with the control group. This finding indicates that the PGE₂ levels were significantly decreased due to inhibition of mPGES-1 by the tested compounds. The results of the dose-dependent study of the leading compound show that the PGE₂ inhibition ability was correlated with the concentration of the compounds. Administration of 10 mg/kg compound could also exert potent anti-inflammation effects as compared with 50 mg/kg Celebrex.

In this study, it is shown that 3-F and 2-Me have significant effects on reducing the paw edema due to acute inflammation in both BABL-c and CD-1 mouse models. These results indicate the potent anti-inflammatory capacity of the leading compounds in vivo. Based on previous high through-put screening and docking studies, 3-F and 2-Me effectively bound to the pocket of the mPGES-1 trimer, but not COX-2 or PGIS, which could possibly explain their potent anti-inflammatory effects. Administration of 50 mg/kg 3-F and 2-Me also decreases the paw thickness, a parameter widely used to evaluate the anti-inflammatory effect of drug candidates [23, 24].

It should be noted that other compounds which inhibit mPGEs-1 using unstable PGH₂ as the substrate have been tested (20,24,25). However, the compounds tested herein have been cross-filtered between mPGES-1, PGIS and COX-2 using stable AA as a substrate under the mimicked COX-2 coupled with mPGES-1 or PGIS in native ER environment. This is an exclusive screening, and the identified lead compounds specifically target mPGES-1. Thus, the compounds with non-crossed inhibition for PGIS and COX-2 are more attractive candidates for the development of a new generation of anti-inflammatory drugs. They can also be substitutes or replacements for current NSAIDs and COX-2 inhibitors, to reduce side effects such as GI insults and increased risk of heart disease.

### REFERENCES

1. Bindu S, Mazumder S, Bandyopadhyay U. Non-steroidal anti-inflammatory drugs (NSAIDs) and organ damage: A current perspective. Biochem Pharmacol. 2020 October; 180:114147. doi: 10.1016/j.bcp.2020.114147. Epub 2020 Jul. 10. PMID: 32653589; PMCID: PMC7347500.
2. Fitzpatrick F A. Cyclooxygenase enzymes: regulation and function. Curr Pharm Des. 2004; 10(6):577-88 doi: 10.2174/1381612043453144. PMID: 14965321.
3. Schjerning A M, McGettigan P, Gislason G. Cardiovascular effects and safety of (non-aspirin) NSAIDs. Nat Rev Cardiol. 2020 September; 17(9):574-584. doi: 10.1038/s41569-020-0366-z. Epub 2020 Apr. 22 PMID: 32322101
4. Simmons, Daniel L, Regina M Botting, and Timothy Hla. 2004. “Cyclooxygenase Isozymes: The Biology of Prostaglandin Synthesis and Inhibition.” Pharmacological Reviews 56 (3): 387-437. doi:10.1124/pr.56.3.3.387.
5. Prostaglandin E synthase. Murakami M, Nakatani Y, Tanioka T, Kudo I. Prostaglandins Other Lipid Mediat. 2002 August; 68-69:383-99. doi: 10.1016/s0090-6980(02)00043-6. PMID: 12432931 Review.
6. Vane, J R, Y S Bakhle, and R M Botting. 1998. “Cyclooxygenases 1 and 2.” Annual Review of Pharmacology and Toxicology 38 (January): 97-120. doi:10.1146/annurev.pharmtox.38.1.97.
7. Advance in understanding the biosynthesis of prostacyclin and thromboxane A2 in the endoplasmic reticulum membrane via the cyclooxygenase pathway. Ruan K H. Mini Rev Med Chem. 2004 August; 4(6):639-47. doi: 10.2174/1389557043403710. PMID: 15279598 Review.
8. Microsomal prostaglandin E synthase (mPGES)-1, mPGES-2 and cytosolic PGES expression in human gastritis and gastric ulcer tissue. Gudis K, Tatsuguchi A, Wada K, Futagami S, Nagata K, Hiratsuka T, Shinji Y, Miyake K, Tsukui T, Fukuda Y, Sakamoto C. Lab Invest. 2005 February; 85(2):225-36. doi: 10.1038/labinvest.3700200. PMID: 15531909.
9. The terminal prostaglandin synthases mPGES-1, mPGES-2, and cPGES are all overexpressed in human gliomas. Mattila S, Tuominen H, Koivukangas J, Stenbäck F. Neuropathology. 2009 April; 29(2):156-65. doi: 10.1111/j.1440-1789.2008.00963.x. MID: 19347995.
10. Microsomal prostaglandin E synthase (mPGES)-1, mPGES-2 and cytosolic PGES expression in human gastritis and gastric ulcer tissue. Gudis K, Tatsuguchi A, Wada K, Futagami S, Nagata K, Hiratsuka T, Shinji Y, Miyake K, Tsukui T, Fukuda Y, Sakamoto C. Lab Invest. 2005 February; 85(2):225-36. doi: 10.1038/labinvest.3700200. PMID: 15531909.
11. Ruan, Ke-He, Hui Deng, and Shui Ping So. 2006. “Engineering of a Protein with Cyclooxygenase and Prostacyclin Synthase Activities That Converts Arachidonic Acid to Prostacyclin.” Biochemistry 45 (47): 14003-11. doi:10.1021/bi0614277.
12. Cardiovascular effects of cyclooxygenase-2 inhibitors: a mechanistic and clinical perspective. Patrono C. Br J Clin Pharmacol. 2016 October; 82(4):957-64. doi: 10.1111/bcp.13048. Epub 2016 Jul. 18. PMID: 27317138 Free PMC article. Review.
13. Membrane prostaglandin E synthase-1: a novel therapeutic target. Samuelsson B, Morgenstern R, Jakobsson P J. Pharmacol Rev. 2007 September; 59(3):207-24. doi: 10.1124/pr.59.3.1. PMID: 17878511 Review.
14. Bacchi S, Palumbo P, Sponta A, Coppolino M F. Clinical pharmacology of non-steroidal anti-inflammatory drugs: a review, Antiinflamm Antiallergy Agents Med Chem. 2012; 11(1):52-64. doi: 10.2174/187152312803476255. PMID: 22934743.
15. Liggett, Jason L, Xiaobo Zhang, Thomas E Eling, and Seung Joon Baek. 2014. “Anti-Tumor Activity of Non-Steroidal Anti-Inflammatory Drugs: Cyclooxygenase-Independent Targets.” Cancer Letters 346 (2). Elsevier Ireland Ltd: 217-24. doi:10.1016/j.canlet.2014.01.021.
16. Structure-based discovery of mPGES-1 inhibitors suitable for preclinical testing in wild-type mice as a new generation of anti-inflammatory drugs. Kai Ding, Ziyuan Zhou, Shurong Hou, Yaxia Yuan, Shuo Zhou, Xirong Zheng, Jianzhong Chen, Charles Loftin, Fang Zheng, Chang-Guo Zhan Sci Rep. 2018; 8: 5205. Published online 2018 Mar. 26. doi: 10.1038/s41598-018-23482-4 PMCID: PMC5979965.
17. Engineering ‘Enzymelink’ for screening lead compounds to inhibit mPGES-1 while maintaining prostacyclin synthase activity. Ruan D T, Tang N, Akasaka H, Lu R, Ruan K H. Future Med Chem. 2021 July; 13(13):1091-1103. doi: 10.4155/fmc-2021-0056. Epub 2021 Jun. 3. PMID: 34080888.
18. Ruan, Ke-He He, Shui-Ping Ping So, Vanessa Cervantes, Hanjing Wu, Cori Wijaya, and Rebecca R. Jentzen. 2008. “An Active Triple-Catalytic Hybrid Enzyme Engineered by Linking Cyclo-Oxygenase Isoform-1 to Prostacyclin Synthase That Can Constantly Biosynthesize Prostacyclin, the Vascular Protector.” The FEBS Journal 275 (23): 5820-29. doi:10.1111/j.1742-4658.2008.06703.x.
19. Morris C J. Carrageenan-induced paw edema in the rat and mouse. Methods Mol Biol. 2003; 225:115-21. doi: 10.1385/1-59259-374-7:115. PMID: 12769480. [carrageenan model]
20. WINTER C A, RISLEY E A, NUSS G W. Carrageenin-induced edema in hind paw of the rat as an assay for andiflammatory drugs. Proc Soc Exp Biol Med. 1962 December; 111:544-7, doi: 10.3181/00379727-111-27849. PMID: 14001233.
21. Nass S R, Steele F F, Ware T B, Libby A H, Hsu K L, Kinsey S G. Monoacylglycerol Lipase Inhibition Using JZL184 Attenuates Paw Inflammation and Functional Deficits in a Mouse Model of Inflammatory Arthritis. Cannabis Cannabinoid Res. 2021 June; 6(3):233-241. doi: 10.1089/can.2020.0177. Epub 2021 May 25. PMID: 34042520; PMCID: PMC8217598.
22. Hanh C h, Khaziakhmetova V N, Ziganshina L E. [MODELING INFLAMMATORY EDEMA: ARE THE MODELS INTERCHANGEABLE], Eksp Klin Farmakol. 2015; 78(7):24-31. Russian. PMID: 26591204.
23. Bai R, Jie X, Sun J, Liang Z, Yoon Y, Feng A, Oum Y, Yu W, Wu R, Sun B, Salgado E, Xie Y, Shim H. Development of CXCR4 modulators based on the lead compound RB-108. Eur J Med Chem. 2019 Jul. 1; 173:32-43. doi: 10.1016/j.ejmech.2019.03.065. Epub 2019 Apr. 5. PMID: 30981691; PMCID: PMC6488424.
24. Lim S M, Jang H M, Jang S E, Han M J, Kim D H. Lactobacillus fermentum IM12 attenuates inflammation in mice by inhibiting NF-κB-STAT3 signalling pathway. Benef Microbes. 2017 May 30; 8(3):407-419. doi: 10.3920331\42016.0156. Epub 2017 May 15. PMID: 28504577.

What is claimed:

1. A method for prevention or treatment of inflammation, inflammatory related diseases disorders, pain or fever, in a subject comprising administering to said subject a compound selected from the group consisting of:
(i) a compound having a modification of the position 1, —OH group (R1) and position 2 —NH group (R2) of 2,4-diaminophenol by 4-(methyl)-(I), 4-(dimethyl)-(II) and 4-(trimethyl)-(III) benzoic acid 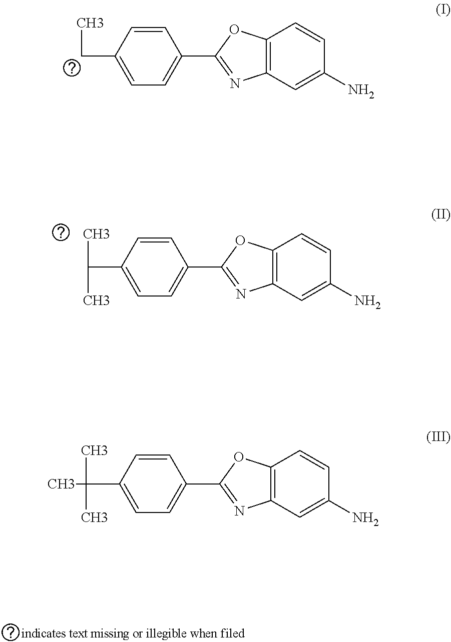 [SMILES: CCc1ccc(-c2nc3cc(N)ccc3o2)cc1 ; CC(C)c1ccc(-c2nc3cc(N)ccc3o2)cc1 ; CC(C)(C)c1ccc(-c2nc3cc(N)ccc3o2)cc1]
(ii) a compound having a modification of the position 1, —OH group (R1) and position 2 —NH group (R2) of 2-amino, 4-nitrol phenol by 4-(methyl)-(IV), 4-(dimethyl)-(V) and 4-(trimethyl)-(VI) benzoic acid 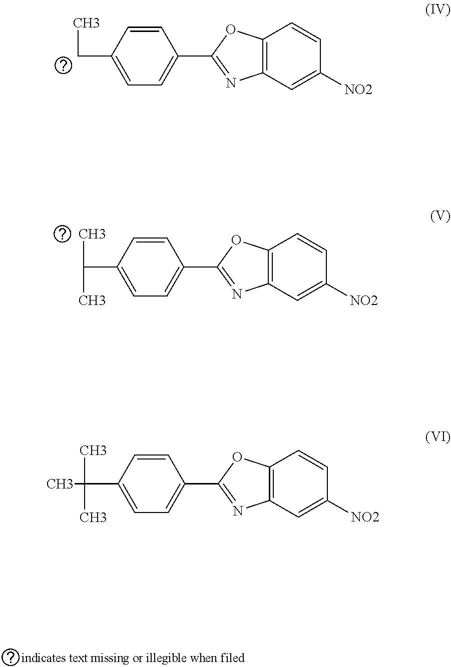 [SMILES: CCc1ccc(-c2nc3cc([N+](=O)[O-])ccc3o2)cc1 ; CC(C)c1ccc(-c2nc3cc([N+](=O)[O-])ccc3o2)cc1 ; CC(C)(C)c1ccc(-c2nc3cc([N+](=O)[O-])ccc3o2)cc1]
(iii) a compound having a modification of the position 1, —OH group (R1) and position 2 —NH group (R2) of 2,4-diaminophenol by 4-(fluoro)-(VII), 4-(difluoro)-(VIII) and 4-(trifluoro)-(IX) benzoic acid 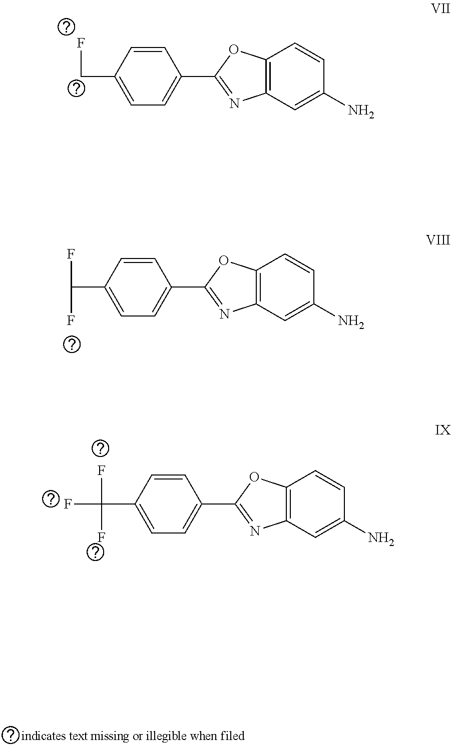 [SMILES: Nc1ccc2oc(-c3ccc(CF)cc3)nc2c1 ; Nc1ccc2oc(-c3ccc(C(F)F)cc3)nc2c1 ; Nc1ccc2oc(-c3ccc(C(F)(F)F)cc3)nc2c1]
and (iv) a compound having a modification of the position 1, —OH group (R1) and position 2 —NH group (R2) of 2-amino, 4-nitrol phenol by 4-(fluoro)-(X), 4-(difluoro)-(XI) and 4-(trifluoro)-(XII) benzoic acid.

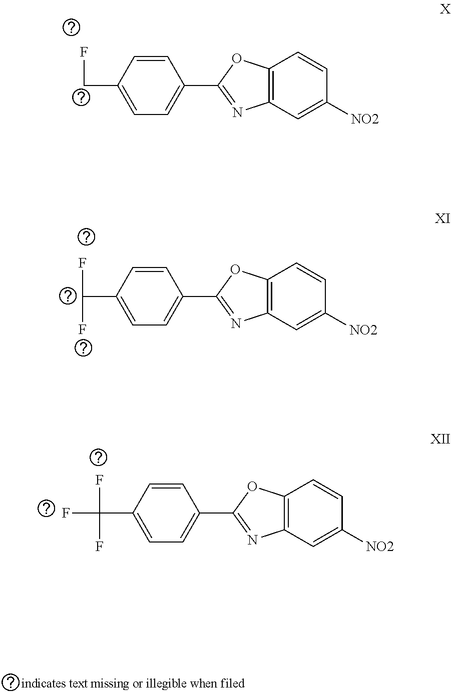 [SMILES: O=[N+]([O-])c1ccc2oc(-c3ccc(CF)cc3)nc2c1 ; O=[N+]([O-])c1ccc2oc(-c3ccc(C(F)F)cc3)nc2c1 ; O=[N+]([O-])c1ccc2oc(-c3ccc(C(F)(F)F)cc3)nc2c1]

2. The method of claim 1 wherein said compound is formulated into a pharmaceutical composition for in vivo administration.

3. The method of claim 2, wherein the pharmaceutical composition is a liquid formulation.

4. The method of claim 2, wherein the pharmaceutical composition is a formulated into a powder, pill, tablet, capsule or any solid and semi-solid form.

5. The method of claim 2, wherein the pharmaceutical composition is a formulated as a nanoparticle, polymer, semi-polymer or combinations thereof.

6. The method of claim 2, wherein the pharmaceutical composition is administered by parenterally, orally, or topically through the skin, or mucosal.

7. The method of claim 6, wherein the parental administration is through an intramuscular, subcutaneous, intravenous, or intradermal injection of the subject.

8. The method of claim 1, for use in the treatment of arthritis.

9. The method of claim 1, for use in the treatment or prevention of pain.

10. The method of claim 9, wherein the pain is caused by endogenous and/or exogenous factors, a viral or bacterial pathogen or associated with cancer, burns, or inflammatory factors.

11. The method of claim 1, for used in the prevention or treatment of autoimmune diseases.

12. The method of claim 1, for used in the prevention or treatment of inflammation associated with cancers.

13. The method of claim 1, for used in the prevention or treatment of inflammation associated with neurodegeneration diseases.

14. The method of claim 1, for used in the prevention or treatment of vascular inflammation diseases.

15. The method of claim 1, for used in the prevention or treatment of inflammation associated with diabetes.

16. The method of claim 1, for used in the prevention or treatment of inflammatory diseases of the skin and hair.

17. The method of claim 1, for used in the prevention or treatment of colitis.
